(19) United States
(12) Patent Application Publication
(10) Pub. No.: US 2023/0093473 A1
(43) Pub. Date: Mar. 23, 2023

(54) SAMPLING KIT FOR THE TRANSPORT OF SPUTUM

(71) Applicants: INSERM (INSTITUT NATIONAL DE LA SANTÉ ET DE LA RECHERCHE MÉDICALE), Paris (FR); CENTRE HOSPITALIER REGIONAL UNIVERSITAIRE, Brest (FR); ETABLISSEMENT FRANCAIS DU SANG, La Plain Saint Denis (FR); UNIVERSITE DE BRETAGNE OCCIDENTALE, Brest (FR); CHU DE RENNES, Rennes (FR)

(72) Inventors: Geneviève HERY-ARNAUD, BREST (FR); Charles-Antoine GUILLOUX, BREST (FR); Claudie LAMOUREUX, BREST (FR)

(21) Appl. No.: 17/904,114

(22) PCT Filed: Feb. 12, 2021
(86) PCT No.: PCT/EP2021/053436

§ 371 Date: Aug. 12, 2022

(30) Foreign Application Priority Data
Feb. 12, 2020 (EP) 20305133.9

(51) Int. Cl. A61B 10/00; A61J 1/12; C12M 1/04

(52) U.S. Cl. CPC A61B 10/0051; A61J 1/12; C12M 1/045

(57) ABSTRACT

There was still an unsatisfied need to have a device specifically adapted for the collection of sputum, for example from CF patients. This technical problem is overcome by the kit of the invention comprising a sterile sampling container having a gas-permeable cap, an anaerobic atmosphere generator, and a sealable pouch adapted to receive the sterile sampling container and the anaerobic atmosphere generator therein. This kit is easy to use, cost effective and makes the transport of the sample easy while the sample can be kept viable for several hours. Our in vitro tests show that the use of the kit improves the number of survival colonies by 3 log after 48 h for V. parvula and by 1 log after 24 h for S. aureus.

### TECHNICAL FIELD

The present invention pertains to the medical field and is of interest in the fight against cystic fibrosis, bronchiectasis, chronic obstructive pulmonary disease (COPD), asthma, or other respiratory diseases that needed microbial analysis.

The present invention makes it possible to transport and keep samples of sputum comprising anaerobic bacteria. The present invention also refers to a method for collecting sputum samples comprising anaerobic bacteria and preserving the sputum in view of a later analysis or transport.

Cystic fibrosis (CF) is a recessive autosomal genetic disease which is caused by mutations of the cystic fibrosis transmembrane conductance regulator (CFTR) gene located on chromosome 7 and coding for the CFTR protein. This CFTR protein is present in the plasma membrane of many mucosa cells (digestive, respiratory, etc.) and codes an ionic channel regulating transport of chlorite anions from one side of the cell membrane to the other. Alteration of the CFTR protein disrupts ion exchange at the mucosa and leads to severe physiochemical and rheological mucous abnormalities, and in particular inhibition of secretion of chloride and increase in sodium absorption at the apical pole of the epithelial cells and decrease of water excretion at the mucosa. Thus, the secreted mucous is more viscous and abundant at the bronchi, pancreatic ducts, intestine, bile duct and vas deferens; which causes the disease symptoms. The mucous thickening leads to non-specific symptoms such as coughing, purulent and mucous expectorations, breathing difficulties, chronic obstructive pulmonary disease (COPD), which can develop into respiratory failure. Bacterial colonisation of the respiratory tree determines the development of the disease. By extension, other inflammatory lung diseases, with or without a genetic-component, that lead to a risk of bronchial and/or pulmonary infection (e.g. bronchiectasis, asthma, COPD) need microbial acute analysis for which anaerobes have to be protected before bacterial culture.

Numerous studies using metagenomics and anaerobic cultures have demonstrated the presence of strict anaerobic bacteria in respiratory specimens from cystic fibrosis (CF), COPD, and asthma patients. Although anaerobic bacteria represent a smaller proportion of the respiratory microbiota than do aerobic bacteria, the density of anaerobic bacteria is estimated to be between 10⁴ and 9×10⁷ colony forming units (CFU) per mL of sputum. The bacterial genera most frequently found by culture in the respiratory tract are Prevotella, Veillonella, Propionibacterium, Peptostreptococcus and Actinomyces.

The metagenomics approach increases the completeness of the description of anaerobic pulmonary bacterial communities by amplification, library construction and high-throughput sequencing of bacterial genomes or a fragment of the bacterial genomes. Metagenomics has thus made it possible to investigate the anaerobic microbiota in depth and to identify anaerobic bacteria that are difficult to cultivate (e.g. Porphyromonas).

The specific role of the anaerobic bacteria in the evolution of the respiratory disease is yet not known. Generally speaking, strict anaerobic bacteria are thought to play a negative role in the evolution of the disease. Strict anaerobic bacteria are also able to cooperate with other microorganisms within the respiratory microbiota and can thus induce virulence of pathogens involved in the degradation of respiratory functions in patients. Strictly anaerobic bacteria are frequently resistant to the antibiotics commonly prescribed to patients. Finally, strict anaerobic bacteria produce numerous proteases, enzymes acting as virulence factors impacting the immune and inflammatory systems.

In contrast, recent studies have demonstrated the beneficial role of anaerobic bacteria in the evolution of respiratory diseases. Indeed, strict anaerobic bacteria could be associated with improved lung function and reduced inflammation. In addition, some species of strict anaerobic bacteria, such as those belonging to the genera Prevotella and Porphyromonas, could be used as biomarkers in the follow-up of CF patients or patients with other chronic pulmonary diseases (e.g. asthma, COPD, bronchiectasis), by modifying their abundance in case of exacerbation of the pulmonary diseases.

Further studies dedicated to investigating the involvement of strict anaerobic species in lung damage and their antibiotic resistance profile are therefore still necessary.

### BACKGROUND ART

It exists in the prior art devices and methods which enable the collect or the culture of anaerobic bacteria. However, they are not adapted for the specific collection of sputum or they are not adapted for the transport of the sample in an anaerobic atmosphere.

U.S. Pat. No. 4,283,498, “Biological specimen collection and transport system”, describes a device adapted for sputum collection. It includes a tubular container and a funnel at one end thereof to collect the sputum. The device is then placed into a sterile envelope for transporting the sputum sample. However, this document does not describe or suggest the use of an anaerobic atmosphere for the transport and storage of the anaerobic bacteria present in sputum. It is therefore impossible to analyze the anaerobic bacteria of interest with this device.

The document U.S. Pat. No. 4,038,148 describes an apparatus for stocking, transporting and testing anaerobic bacterial cultures. Such an apparatus is however not directed to the sampling of sputum. Furthermore, such an apparatus comprises an ampoule in combination with a desiccant pellet to generate the anaerobic atmosphere. As U.S. Pat. No. 4,038,148, the document JP2016028555 is not adapted for collecting sputum and comprises an ampoule that must be broken to generate the anaerobic atmosphere.

U.S. Pat. Nos. 4,108,728 and 4,014,748 are two other documents related to an apparatus for transporting bacteria in an anaerobic atmosphere. Such apparatus are however not adapted for the collection and transporting of sputum. U.S. Pat. No. 3,913,564, “Anaerobic specimen collecting and transporting device”, describes a method and a device for collection and transport of anaerobic bacteria. The device consists of a cotton swab and a tube with a frangible ampule. The liquid in the frangible ampule reacts with the oxygen present in the tube to make the atmosphere in the tube anaerobic. However, such a device is complex to implement and makes the collection of the sample uncomfortable for the patient. It is furthermore not suitable for the specific collection of sputum. Moreover, the frangible ampule in U.S. Pat. No. 3,913,564 is not reusable and the device is thus not sustainable.

CN105420073, “Anaerobe culture bag and method for culturing anaerobe by using anaerobe culture bag”, describes a kit for culturing anaerobic bacteria with a pouch and Petri dishes. However, said pouch has a valve to create an air vacuum, making the use of this kit more complex and not specifically adapted for the transport of the samples. Such a device is furthermore not adapted for the specific collection and transport of sputum.

### Technical Problem

Therefore, there was still an unsatisfied need to have a device specifically adapted for the collection of sputum, for example from CF patients. Furthermore, it is crucial to preserve the anaerobic atmosphere of the collected sample during its transport, for example from the collecting place to an analysis laboratory which may be distant from the collecting place.

Finally, collecting sputum from a patient can be complicated for the healthcare professional and unpleasant for the patient. It is therefore necessary to develop a fast, efficient and easy to use mean that would allow sputum to be collected and transported while ensuring the viability of the sample.

### SUMMARY OF THE INVENTION

The inventors have thus developed a kit for collecting and transporting a sputum sample of a patient comprising:
a sterile sampling container having a gas permeable cap,
an anaerobia atmosphere generator, and
a sealable pouch adapted to receive the sterile sampling container and the anaerobic atmosphere generator therein.

Sputum samples require adapted transportation system regarding some specificities described below. Sputum samples are collected spontaneously or after induction from patients with cystic fibrosis or suffering from other diseases like asthma, chronic obstructive pulmonary disease, and from healthy subjects, as well.

Sputum samples are precious biological material as they may be used for patients' follow-up, diagnosis or medical research purposes.

Sputum samples are usually obtained in limited volume, especially induced sputum samples (from few microliters (μL) to generally less than one milliliter (mL)). Sputum is a more complex sample than blood, tissue or urine as it may be highly viscous, heterogeneous, and it may contain complex microorganisms communities, including anaerobic bacteria.

Regarding these features, the transportation of sputum samples needs specific precautions. An anaerobic atmosphere is required to protect anaerobes as oxygen may rapidly reach the entire sample when sputum is composed of few microliters (μL). Sputum samples may be used for microbiological patients' follow-up and shouldn't be in contact with external sources of contamination. As the quantity of sample is low, contamination may be observed in subsequent analysis.

A sterile sampling container with a gas permeable cap avoids any contamination of the sputum while gas exchange is available allowing the anaerobic atmosphere to establish.

The kit is easy to use, cost effective and makes the transport of the sample easy while the sample can be kept viable for several hours.

The kit of the present invention may be used for keeping samples containing anaerobic bacteria or strictly anaerobic bacteria viable.

Non exhaustive examples of strictly anaerobic bacteria are listed in the following table (in particular Prevotella, Veillonella, Propionibacterium, Peptostreptococcus and Actinomyces).

| Negative gram | | Positive gram | |
|---|---|---|---|
| Cocci | Bacillus | Cocci | Bacillus |
| Veillonella | Bacteroides | Peptostreptococcus | Actinomyces |
| (Firmicutes) | (Bacteroidetes) | (Firmicutes) | (Actinobacteria) |
| Megasphaera | Prevotella | Parvimonas | Propionibacterium |
| (Firmicutes) | (Bacteroidetes) | (Firmicutes) | (Actinobacteria) |
| | Porphyromonas | | Bulleidia |
| | (Bacteroidetes) | | (Firmicutes) |
| | Fusobacterium | | Clostridium |
| | (Fusobacteria) | | (Firmicutes) |
| | Tannerella | | Eubacterium |
| | (Bacteroidetes) | | (Firmicutes) |
| | Leptotrichia | | Lachnospiraceae |
| | (Fusobacteria) | | (Firmicutes) |
| | Catonella | | Mogibacterium |
| | (Firmicutes) | | (Firmicutes) |
| | Dialister | | Atopobium |
| | (Firmicutes) | | (Actinobacteria) |
| | Selenomonas | | Solobacterium |
| | (Firmicutes) | | (Firmicutes) |
| | Johnsonella | | |
| | (Firmicutes) | | |

The sealable pouch is preferably made of a material impermeable to oxygen, for example the sealable pouch is made of a plastic material such as polyolefin, e.g. polyethylene.

A sealable pouch impermeable to oxygen increases the viability duration of the sample in combination with the anaerobia atmosphere generator. For example, the sealable pouch comprises an oxygen-barrier layer.

The sealable pouch may be flexible. Such a pouch is more convenient to be transported and can adapt to different geometries of containers.

The sealable pouch may be transparent so that it is possible to visually check the sample without opening the pouch.

The sealable pouch may be made of one single piece of material or several pieces of a same material. For example, the pouch is made of two sheets that are heat-welded in their edges except a portion thereof to form a pouch.

The sealable pouch may also be made of different materials. For example, the sealable pouch is made of two sheets of two different materials. The two sheets are heat welded in their edges except a portion thereof so as to form a pouch. One sheet may be transparent and the other sheet may be non-transparent (for example tinted). The material may also be chosen to make it possible to write onto it using a pencil or a ball pan or to easily stick one tag onto it.

Alternatively, a bellows can be provided between the two sheets of same or different materials. This makes it possible to increase the capacity of the pouch. The bellows may have a single fold or multiple folds.

The capacity is understood as referring to the maximum volume that the sealable pouch can contain. The capacity of the sealable pouch is for example comprised between 1 L and 10 L.

The sealable pouch is preferably a quadrilateral shape, for example a rectangular, with one side being open. One width is preferably open but it could also be the length. Also, more than only one side could be open and then heat sealed or sealed with a specific sealer as described above.

The sealable pouch may comprise a sealer enabling sealing and unsealing the sealable pouch without destruction of the sealable pouch.

Such a sealer enables to easily open and close the sealable pouch while the atmosphere inside the pouch is kept anaerobic when the sealable pouch is closed. Moreover, the use of a sealer enabling sealing and unsealing the sealable pouch without destruction of the sealable pouch makes it possible to reuse the sealable pouch.

It is easily understandable that the reuse of a sealable pouch can be done provided that the sealable pouch is well cleaned and/or sterilized according to known methods.

The sealer may advantageously comprise a tube with a slit extending from one end of the tube to the other end thereof and a cylinder slidably fittable inside the tube along the length thereof. Such a sealer makes it easy to seal and unseal the pouch by pinching the pouch between the tube and the cylinder. It can be reused and easily cleaned and/or be sterilized.

The sealer preferably seals one side of the pouch. Also, several sealers may be used.

The sealer may comprise on a first inner side of the pouch a groove with flexible walls on both side of the groove and, on a second inner side of the pouch facing the first inner side, a rail member adapted to the groove to be locked therein by pressure. Sealing may be done by hand, pressing the sides to one another so that the rail fits into the groove. The sealer can also comprises a cursor placed astride on both groove and rail and adapted to apply a pressure to the groove and rail towards one another when biased in one direction and to release the pressure when biased in the opposite direction.

The anaerobic atmosphere generator may be a solid anaerobic gas generator, optionally contained in a sachet.

A solid anaerobic gas generator, optionally contained in a sachet, is easy to handle and can be kept for a long time until it is needed.

The anaerobic gas replaces the air, especially the dioxygen (O₂) contained inside the pouch. The anaerobic gas is for example nitrogen (N₂) or preferably carbon dioxide (CO₂).

The sachet may be made in a porous material such as a tea bag. The sachet may be kept in a gas-impermeable packaging. It would therefore be possible to keep the anaerobic gas generation capacity of the solid anaerobic gas generator as long as it is kept inside the packaging. And as soon as the sachet is taken out from the packaging, the solid anaerobic gas generator consumes air and provides an anaerobic atmosphere.

The solid anaerobic gas generator capacity may be chosen according to the pouch capacity or the container capacity. The solid anaerobic gas generator may comprise ascorbic acid and activated carbon as active components; i.e. components that will react with air (in particular O₂) to generate anaerobic gas, preferably CO₂.

For instance, a CO₂ generator may be a Sachet Oxoid™ AnaeroGen™ Compact sold by Thermofischer.

The sterile sampling container may be made of glass such as transparent or opaque glass. The sterile sampling container may also be made of thermoplastic such as polypropylene. Such materials are easy to clean and/or sterilize so that the sterile sampling container may be reused.

The sterile sampling container needs to have a sufficient capacity for collecting sufficient sputum in view of the later analysis. However, the sampling container does not have to be too big because the viability of the sample would decrease and it would be more difficult to handle.

The sterile sampling container may have a capacity comprised between 5 and 100 mL, preferably between 25 and 75 mL.

As the sterile sampling container may have a capacity comprised between 5 and 100 mL, preferably between 25 and 75 mL, patient can spit directly in the sampling container, avoiding any contamination or sample manipulation.

The sterile sampling container may be a tube. One end of the tube may be in the form of a truncated cone.

The tube may advantageously be graduated, for example having one or several lines indicating the filled volume of the tube like a measuring glass.

The sterile sampling container is for example the CELLSTAR CELLreactor® tube sold by Greiner.

The sampling container is closed with a gas-permeable cap. The gas permeable cap retains the sputum sample inside the sampling container while the inside of the sampling container can still be in gas-communication with the inside of the pouch, thus enabling oxygen within the sampling container to travel out of the sampling container and contact the anaerobic atmosphere generator; or for the anaerobic atmosphere generator (when this latter is a gas) to travel form the inside of the pouch into the inside of the sampling container and consume the oxygen present therein.

The gas permeable cap may have a porous membrane. The gas permeable cap could also comprise holes which are sufficiently small to keep the sputum inside the container but sufficiently large to be oxygen permeable.

In another embodiment, the gas permeable cap may be a combination of a solid cap comprising through holes with a permeable membrane.

The gas permeable cap is advantageously a screwable cap. A screwable cape makes it easier to open and close the sampling container while the closing is secured. Even during the transport the sampling container does not risk to be involuntary opened.

The kit may further comprise a sterile pipet, a sterile spit jar or several sterile sampling containers with different capacities. This is particularly useful when the patient cannot expectorate multiple times (as it is sometimes necessary to proceed to standard bacteriological, mycobacteriological and mycological tests with one expectoration per test) and sputum has to be collected in the sterile spit jar before being transferred into the sterile sampling container by pipetting.

The kit may further comprise additives. For example the additive may be a culture medium enabling proliferation of the bacteria. The additive may also be a liquefying agent, for example dithiothreitol (IUPAC name: (2S,3S)-1,4-Bis-sulfanylbutane-2,3-diol). The additive may also be an additive enabling selection of specific species of bacteria, for example an antibiotic to select only the bacteria which are resistant to the antibiotic.

The kit may further comprise an indicator dye such as resazurin (IUPAC name: 7-hydroxy-10-oxidophenoxazin-10-ium-3-one) strip. The indicator dye can thus be introduced inside the pouch and serve as an indicator of the anaerobic atmosphere within the pouch.

Quantitative PCR primers and probes for the detection and quantification of at least one genus of bacteria, for example the genera Fusobacterium, Prevotella and Veillonella and the species Porphyromonas catoniae and Porphyromonas endodontalis may be used after the collect of the sputum with the kit of the present invention.

The inventors have also developed a method for collecting a sputum sample from a patient, comprising the steps of:
collecting the sputum sample in a sterile sampling container,
closing the sterile sampling container with a gas permeable cap,
enclosing the closed sterile sampling container and an anaerobic atmosphere generator into a sealable pouch, and
sealing the sealable pouch.

The method of the present invention makes the collect of a sputum sample easier for the healthcare professional and more convenient for the patient. Indeed, the patient can directly expectorate into the sterile sampling container.

The collection of the sputum may be done in an anaerobic atmosphere. However, the present invention makes it possible to collect the sputum in an aerobic atmosphere and then place said collected sputum in the inside of the sealable pouch of the present invention with the anaerobic atmosphere generator to create an anaerobic atmosphere around the sample.

The collected sputum may be mixed with additives. For example the additive may be a culture medium enabling the proliferation of the bacteria. The additive may also be liquefying agent, for example dithiothreitol. The additive may also be an additive enabling the selection of specific bacteria, for example an antibiotic to select only the antibiotic resistant bacteria.

The enclosing step may comprise first placing the sterile sampling container comprising the sputum sample inside the sealable pouch and then placing the anaerobic atmosphere generator inside the sealable pouch. Thus, the anaerobic atmosphere generator is close to the sterile sampling container so that oxygen around and inside it is first consumed by the anaerobic atmosphere generator. Providing the anaerobic atmosphere generator above the sterile sampling container containing the sputum sample increases the viability duration of the anaerobes in the sample.

Before the enclosing step, as much as possible air should preferably be extracted from the inside of the sealable pouch. For example, by simply hand-pressing the pouch before sealing or by using a vacuum pump after sealing.

In the case that vacuum is made using a vacuum pump, the sealable pouch advantageously comprises a valve. The air comprised inside the sealable pouch may be pumped through the valve. Once pumped, the valve stays close so that no air re-enters the inside of the sealable pouch. This can be achieved for example by providing a one-way valve.

The sealing step may comprise sealing the pouch with a sealer enabling sealing and unsealing of the sealable pouch without destruction thereof.

Such a step makes it possible to collect a plurality of sputum sample. For example, if two sputum samples need to be collected, one can be collected and then enclosed into the sealable pouch in an anaerobic atmosphere. The second sputum collect can be done while the first sputum collect is kept viable in an anaerobic atmosphere. Once the second sputum sample is collected, it can be easily enclosed in the same pouch as the first one without destruction of the pouch thanks to the aforementioned sealer.

In another embodiment, the pouch can also be heat welded. A heat welded pouch may be more impermeable to oxygen but destruction of the pouch will be necessary if it has to be opened.

Collecting of the sputum sample in a sterile sampling container may further comprise first collecting the sputum in a sterile spit jar, and then transferring the collected sputum into the sterile sampling container with a sterile pipet.

The patient may have some difficulties for expectorating multiple times. In that case, the patient could expectorate first in a sterile spit jar. Then, the sputum sample may be transferred from the sterile spit jar into the sterile sampling container with a sterile pipet. This makes it possible to have several tests being carried out for the patient.

It is worth to note that the device and the method of the present invention have specifically been developed with the objective of collecting sputum from CF patient for the research on CF. However, the skilled person will unambiguously understand that the device and the method of the present invention can be used whatever the sample to collect and the anaerobia bacteria to be analyzed are.

### BRIEF DESCRIPTION OF DRAWINGS

Other features, details and advantages will be shown in the following detailed description and on the illustrative and non-limiting figures:

FIG. 1 is a scheme of an open sealable pouch comprising a sterile sampling container having a gas-permeable cap and an anaerobic atmosphere generator.

FIG. 2 is a scheme of a sealed pouch comprising a sterile sampling container having a gas-permeable cap and an anaerobic atmosphere generator.

FIG. 3 is an example of a sealer according to the present invention.

FIG. 4 is a graph showing the quantification of Veillonella parvula colonies (in UFC/mL) in a sampling container preserved in anaerobic or aerobic atmosphere during 48 h, in comparison with the quantification before the bacteria are placed in the sampling container (initial counting at t=0).

FIG. 5 is a graph showing the quantification of Staphylococcus aureus colonies (in UFC/mL) in a sampling container preserved in anaerobic or aerobic atmosphere during 24 h, in comparison with the quantification before the bacteria are placed in the sampling container (initial counting at t=0).

### DESCRIPTION OF EMBODIMENTS

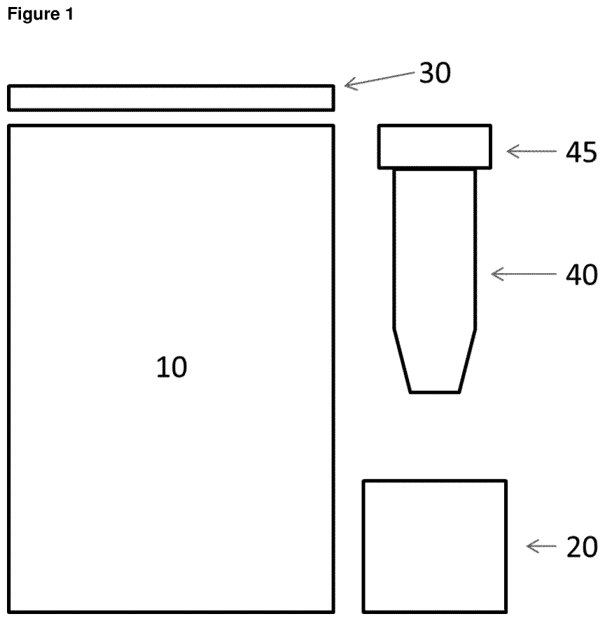

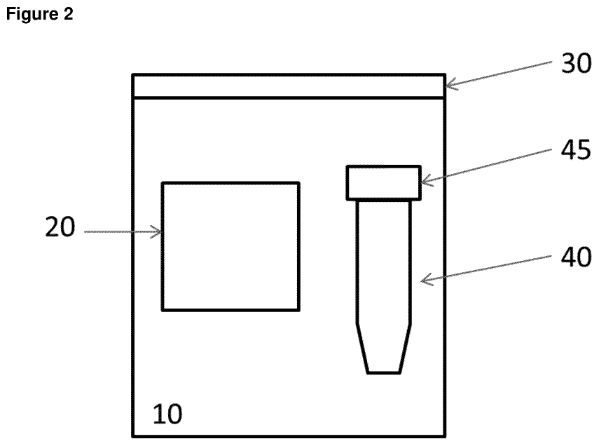

A kit for collecting and transporting a sputum sample of a patient is now described in detailed with regard to FIGS. 1 and 2.

The kit comprises:
a sterile sampling container 40 having a gas-permeable cap 45,
an anaerobic atmosphere generator 20, and
a sealable pouch 10 adapted to receive the sterile sampling container 40 and the anaerobic atmosphere generator therein 20.

The sterile sampling container 40 is a tube of 50 mL made of polypropylene. It has on its side a plurality of lines indicating the capacity of the tube. One end of the tube has a decreasing diameter so that one end of the tube is a truncated cone.

The gas-permeable cap 45 is a solid cap with holes on its upper face. The gas-permeable cap 45 is also a screwable cap. The screwable cap 45 is screwed on the tube on the opened side opposite to the truncated cone.

The sealable pouch 10 is transparent, flexible and made of plastic. The sealable pouch 10 has a rectangular geometry with one width being open.

The opened side of the sealable pouch 10 is sealed with a sealer 30 in the form of a tube.

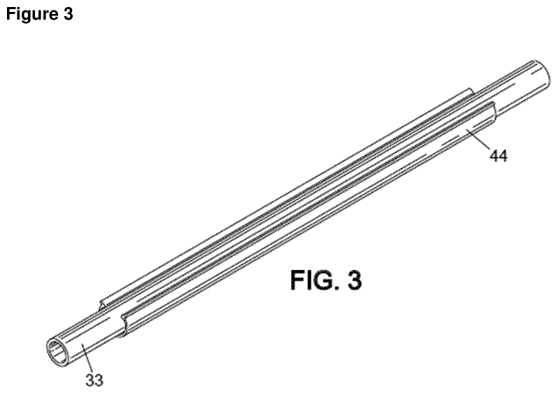

It is now made reference to the FIG. 3. The sealer 30 comprises a tube 44 with a slit extending from one end of the tube 44 to the other end thereof and a cylinder 33 fitted inside the tube 44. To close the sealable pouch 10, the sealable pouch 10 is wrapped around the cylinder 33. The cylinder 33 is then placed inside the tube 44 so that the sealable pouch is sealed.

The anaerobic atmosphere generator 20 is a CO₂ generator contained in a sachet made of a porous material. The anaerobic atmosphere generator 20 is kept in a metallic packaging avoiding any contact between the anaerobic atmosphere generator 20 and air. Depending on the anaerobic atmosphere generator used, the sachet may reduce the oxygen content in the pouch to below 1% within 30 minutes. The resulting carbon dioxide content will be between 8 and 14%.

### Qualitative Validation of the Sampling Kit

A qualitative validation of the kit according to the preferred embodiment of the present invention was done to ensure that an anaerobic atmosphere is successfully generated in the pouch.

Strips of resazurin (a colored redox indicator which is pink when in contact with oxygen) are placed in ten sterile sampling containers.

Each of the ten containers is closed with a screwable gas permeable cap. The closed containers comprising the strip of resazurin are then placed in sealable pouches.

Anaerobic atmosphere generators are then placed in each pouch. The ten pouches are finally sealed with sealers as the one shown in FIG. 2.

The strips of resazurin disposed in the kit have lost their pink colour approximately 3 hours after the pouch was sealed.

Thus, the kit can be effectively used for the transport of sputum sample.

### Quantitative Validation of the Sampling Kit

A quantitative validation of the kit according to the preferred embodiment of the present invention was done to ensure that the sample can be successfully kept viable for several hours.

Defined number of colonies of strictly anaerobic bacteria V. parvula and aero-anaerobic S. aureus are placed into a sterile sampling container closed with a gas permeable cap. The closed container is then placed with the anaerobic atmosphere generator into a pouch. The pouch is finally sealed with a sealer as shown in FIG. 2.

The colonies of strict anaerobic bacteria V. parvula are kept for 48 hours in the anaerobic atmosphere within the sealable pouch.

After 48 h, counting of the colonies of bacteria according to usual method is done to check the viability of the anaerobic atmosphere inside the sealed pouch.

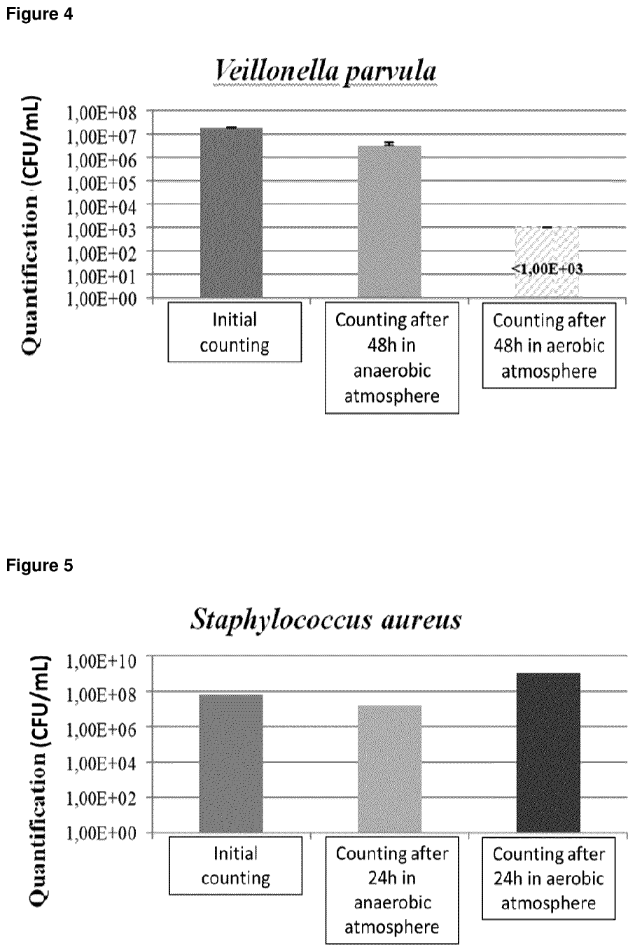

As it can be seen in FIG. 4, after 48 h in the kit according to an embodiment of the present invention, the number of colonies slightly decrease from 1 E+07 UFC/mL to approximately 1.5E+06 UFC/mL. In contrast, the number of colonies drastically decreases after 48 h in the kit in an aerobic atmosphere from 1 E+07 UFC/mL to approximately 1 E+03 UFC/mL. Therefore, the loss of bacteria was only 1 log with the kit of the invention and 4 log otherwise.

It has thus been demonstrated that the present invention makes it possible to keep a sputum sample comprising strict anaerobic bacteria viable for at least 48 h.

The colonies of aero-anaerobic bacteria S. aureus are kept for 24 hours in the anaerobic atmosphere within the sealable pouch.

As it can be seen in FIG. 5, after 24 h in the kit according to an embodiment of the present invention, the loss of bacteria was only 1 log. In contrast, the increase of the number of colonies after 24 h in an aerobic atmosphere was 2 log.

It has thus been demonstrated that the present invention makes it possible to stop the proliferation of aero-anaerobic bacteria and keeps a sputum sample comprising aero-anaerobic bacteria viable for at least 24 h.

1. A kit for collecting and transporting a sputum sample of a patient comprising:
a sterile sampling container having a gas-permeable cap,
an anaerobic atmosphere generator, and
a sealable pouch configured to receive the sterile sampling container and the anaerobic atmosphere generator therein.

2. The kit according to claim 1, wherein the sealable pouch is made of plastic and is impermeable to oxygen.

3. The kit according to claim 1, further comprising a sealer configured to seal and unseal the sealable pouch without destruction of the sealable pouch.

4. The kit according to claim 3, wherein the sealer comprises a tube with a slit extending from one end of the tube to the other end thereof and a cylinder fitted inside the tube.

5. The kit according to claim 1, wherein the anaerobic atmosphere generator is a solid CO₂ generator, optionally contained in a sachet.

6. The kit according to claim 1, wherein the sterile sampling container has a capacity comprised between 5 and 100 mL.

7. The kit according to claim 1, wherein the sterile sampling container is made of thermoplastic.

8. The kit according to claim 1, wherein the gas permeable cap is a screwable cap.

9. A method for collecting a sputum sample of a patient, comprising the steps of:
collecting the sputum sample in a sterile sampling container,
closing the sterile sampling container with a gas permeable cap,
enclosing the closed sterile sampling container and an anaerobic atmosphere generator in a sealable pouch, and
sealing the sealable pouch.

10. The method according to claim 9, wherein the step of enclosing step comprises first placing the sterile sampling container comprising the sputum sample inside the sealable pouch and then placing the anaerobic atmosphere generator inside the sealable pouch thereafter.

11. The method according to claim 9, wherein the step of sealing comprises sealing the pouch with a sealer which seals and unseals the sealable pouch without destruction thereof.

12. The method according to claims 9, wherein the step of collecting further comprises collecting the sputum in a sterile spit jar, and transferring the collected sputum into the sterile sampling container with a sterile pipet.

13. The kit according to claim 6, wherein the sterile sampling container has a capacity of from 25 to 75 mL.

14. The kit according to claim 7, wherein the thermoplastic is polypropylene.
